(19) United States
(12) Patent Application Publication
(10) Pub. No.: US 2022/0128729 A1
(43) Pub. Date: Apr. 28, 2022

(54) METHOD AND DEVICE FOR ESTIMATING MECHANICAL PROPERTY OF ROCK JOINT

(71) Applicant: AALTO UNIVERSITY FOUNDATION SR, Aalto (FI)

(72) Inventors: Lauri UOTINEN, Espoo (FI); Jyrki ORASKARI, Espoo (FI)

(21) Appl. No.: 17/426,781

(22) PCT Filed: Jan. 30, 2020
(86) PCT No.: PCT/FI2020/050052

§ 371 Date: Jul. 29, 2021

(30) Foreign Application Priority Data
Jan. 30, 2019 (FI) 20195061

(51) Int. Cl. G01V 8/02; G01C 3/08; G01B 11/30; G01B 11/26; G01C 11/06; G01C 11/02; G01N 33/24

(52) U.S. Cl. CPC G01V 8/02; G01C 3/08; G01B 11/303; G01B 2210/52; G01C 11/06; G01C 11/02; G01N 33/24; G01B 11/26

(57) ABSTRACT

A method and devices for estimating a mechanical property of a rock joint are presented. The method comprises obtaining a plurality of at least partly overlapping photos of the rock joint by at least one optical sensor comprised in a device, wherein said photos represents the rock joint from different perspectives or positions, generating a digital three-dimensional representation of the rock joint based on said plurality of photos, and determining the mechanical property of the rock joint based on the generated digital three-dimensional representation

### FIELD OF THE INVENTION

The present invention relates in general to devices and methods for evaluating the strength or stability of rock mass. In particular, however not exclusively, the present invention concerns devices and methods for determining a mechanical property of a rock joint, such as roughness.

### BACKGROUND

There are more than hundreds of large open pit mines currently in operation globally. Stability of the rock mass is an essential requirement in these open pit mining operations. The strength of the rock mass is heavily influenced by the rock joints crossing it, and rock joint roughness is one of the factors determining the stability of the rock joint.

Open pit mining includes large excavation in surface of earth with depths exceeding 400 meters. These large-scale excavations result in large pit walls and benches. Collapse of these benches currently forms one of the most severe form of geotechnical accidents in open pit operations. Due to the scale of the mining operation, these collapses can result in millions of cubic metres of earth moving down the pit violently causing damage to life and property.

Fractures in the mining surface are one of the most common failure sources in bench collapse incidents. Properties of these fractures such as their roughness, filling, extent etc. decide how weak the wall is.

Known solutions are based on subjective, manual point-like measurements. There measurements are then converted to an empirical correction factor, such as the roughness factor, and, finally, safety factors are added to handle uncertainties. Thus, the mining operator typically sends a person to the site to manually and subjectively select a surface of interest, measure it with a manual profilometer with sliding metal spikes and then subjectively compare the obtained profile to a standard set of curves and select the best match visually.

The known solutions, however, produce, not only vague results but also involves a lot of manual labour and time to cover a large area. The results are highly variable, unpredictable and typically poorly representative forcing the mining operators to use large safety factors to compensate. This leads to significant ore losses.

### SUMMARY

An objective of the present invention is to provide a method and a device for determining a mechanical property of a rock joint, and a handheld device, an unmanned aerial vehicle comprising the device, and a combination of the device and a rotatable platform. Another objective of the present invention is that the method and the devices at least alleviate some of the drawback in the known solutions, such as produce more accurate results faster.

The objectives of the invention are reached by a method, a device, a handheld device, an unmanned aerial vehicle and a combination of the device and a rotatable platform as defined by the respective independent claims.

According to a first aspect, a method for estimating a mechanical property of a rock joint is provided. The method comprises
obtaining a plurality of at least partly overlapping photos of the rock joint by at least one optical sensor comprised in a device, wherein said photos represents the rock joint from different perspectives or positions,
generating a digital three-dimensional (3D) representation of the rock joint based on said plurality of photos, optionally, by a control unit of the device, and
determining the mechanical property of the rock joint based on the generated digital three-dimensional representation.

In various embodiments, the mechanical property may be roughness, shear strength or friction angle. In another embodiment, the mechanical property may be roughness and shear strength, or roughness and friction angle, or shear strength and friction angle. In still further embodiment, the mechanical property may be roughness, shear strength and friction angle.

In various embodiments, a number of said plurality of photos of the rock joint may be at least 20 or at least 40 photos. Alternatively, the number may be in the range of 20-40 photos. Alternatively or in addition, the number may be at least 100.

In various embodiments, the generation of the digital three-dimensional representation includes determining a point cloud.

In general, the point cloud is a set of data points in space. As referred to herein, the point cloud may be produced by the at least one optical sensor, and optionally measurement data by at least one distance sensor, which determines number of points on the external surface of the rock joint or the surface thereof. The point cloud may then in accordance with the present invention transformed or converted to the digital three-dimensional representation of the rock joint for determining the mechanical property of the rock joint.

In various embodiments, said plurality of photos may represent the rock joint from different angles with respect to the rock joint, such as 10, 20, 45, 60, 75 or 90 degrees.

In various embodiments, the device may comprise a distance sensor, such as an infrared distance sensor, and the method may comprise providing instructions based on measurement data of the distance sensor to an operator via a display of the device for obtaining the plurality of at least partly overlapping photos.

The instructions may include providing the distance of the device from the rock joint, for instance. Alternatively or in addition, the instructions may include providing details about moving the device with respect to the rock joint, such as based on angles when moving in rotatory manner about the rock joint, or amount of centimetres or metres when moving linearly with respect to the rock joint, such as moving past the rock joint.

In various embodiments, the method may comprise moving the optical sensor with respect to the rock joint while the rock joint remains still in its position. Furthermore, said at least one optical sensor may be comprised in a handheld device, wherein the method may comprise moving the handheld device for obtaining the plurality of photos. In a further embodiment, said at least one optical sensor may be comprised in an unmanned aerial vehicle, such as a quadcopter, wherein the method may comprise moving the unmanned aerial vehicle for obtaining the plurality of photos.

In various embodiments, the method may comprise moving the rock joint with respect to said at least one optical sensor while said at least one optical sensor remains still in its position. Alternatively or in addition, the rock joint may be included in a sample arranged on a rotatable platform, wherein the method comprises moving the rock joint by the rotatable platform, such as by rotating the platform around its rotation axis.

In various embodiments, the method may comprise determining overlapping parts of said plurality of photos, and generating the digital three-dimensional representation of the rock joint based on the overlapping parts.

In various embodiments, the method may comprise removing unnecessary points of the point cloud, such as points outside a portion comprising the rock joint. The portion may be, for example, the area on the surface which is limited by a distance of 50 centimetres from the rock joint.

In various embodiments, the method may comprise performing a triangulation of the point cloud, such as for generating the digital three-dimensional representation.

According to a second aspect, a device for estimating a mechanical property of a rock joint is provided. The device comprises at least one optical sensor, such as a camera or cameras, for obtaining photos of the rock joint, and a control unit in connection with the at least one optical sensor, wherein the control unit comprises a processing unit, such as a processor or a microcontroller, configured to
generate a digital three-dimensional representation of the rock joint based on plurality of at least partly overlapping photos of the rock joint, and
determine the mechanical property of the rock joint based on the generated digital three-dimensional representation.

In various embodiments, the device may be configured to obtain the at least partly overlapping photos of the rock joint. The number of photos may be at least 20 or at least 40 photos. Alternatively, the number may be in the range of 20-40 photos. Alternatively or in addition, the number may be at least 100.

In various embodiments, the device may comprise a distance sensor, such as an infrared distance sensor. Alternatively or an addition, the processing unit may be configured to provide instructions based on measurement data of the distance sensor to an operator via a display of the device for obtaining the plurality of at least partly overlapping photos, such as related to moving said device between two photos.

According to a third aspect, a handheld device comprising the device according to the second aspect is provided. The handheld device may comprise a display via which instructions may be given to the operator or user for obtaining the photos of the rock joint, such as related to moving said device between two photos.

According to a fourth aspect, an unmanned aerial vehicle, such as a quadcopter, comprising the device according to the second aspect is provided. The unmanned aerial vehicle preferably comprise means for flight and for controlling the movement of the vehicle, such as by predetermining the flight route, or by a remote control.

According to a fifth aspect, a combination of a rotatable platform and the device according to the second aspect is provided. The rotatable platform may comprise a motor, such as a servomotor, for rotating the platform, such as a table, in controlled manner. The rotation may be controlled to implemented by a desired angle at a time, that is between two photos.

The present invention provides a method and a device for determining a mechanical property of a rock joint, and a handheld device and an unmanned aerial vehicle comprising said device, and a combination of said device and a rotatable platform. The present invention provides advantages over known solutions in that the method and devices are both faster and has much better accuracy than in the known solutions. This allows to cover larger areas faster than before or to scan more often than previously. The present invention enables fast and accurate measurement compared to less accurate and slower manual measurements. By more accurate results ore losses can be avoided in mining operations, for instance.

Various other advantages will become clear to a skilled person based on the following detailed description.

The expression “a number of” may herein refer to any positive integer starting from one (1), that is, being at least one.

The expression “a plurality of” may refer to any positive integer starting from two (2), respectively, that is, being at least two.

The terms “first”, “second” and “third”, for instance, are herein used to distinguish one element or embodiment from other element, and not to specially prioritize or order them, if not otherwise explicitly stated.

The exemplary embodiments of the present invention presented herein are not to be interpreted to pose limitations to the applicability of the appended claims. The verb “to comprise” is used herein as an open limitation that does not exclude the existence of also un-recited features. The features recited in depending claims are mutually freely combinable unless otherwise explicitly stated.

The novel features which are considered as characteristic of the present invention are set forth in particular in the appended claims. The present invention itself, however, both as to its construction and its method of operation, together with additional objectives and advantages thereof, will be best understood from the following description of specific embodiments when read in connection with the accompanying drawings.

### BRIEF DESCRIPTION OF FIGURES

Some embodiments of the invention are illustrated by way of example, and not by way of limitation, in the figures of the accompanying drawings.

FIG. 1 illustrates schematically a device according to an embodiment of the present invention.

FIG. 2 illustrates a flow diagram of a method according to an embodiment of the present invention.

FIG. 3 illustrates schematically a principle for determining the mechanical property of the rock joint in accordance with an embodiment of the present invention.

FIGS. 4A-4C illustrate different perspectives or positions of the optical sensor with respect to the rock joint in accordance with some embodiments of the present invention.

FIG. 5 illustrates schematically a handheld device according to an embodiment of the present invention.

FIG. 6 illustrates schematically an unmanned aerial vehicle according to an embodiment of the present invention.

FIG. 7 illustrates schematically a combination of a device and a rotatable platform according to an embodiment of the present invention.

FIG. 8 illustrates schematically a control unit according to an embodiment of the present invention.

### DETAILED DESCRIPTION OF SOME EMBODIMENTS

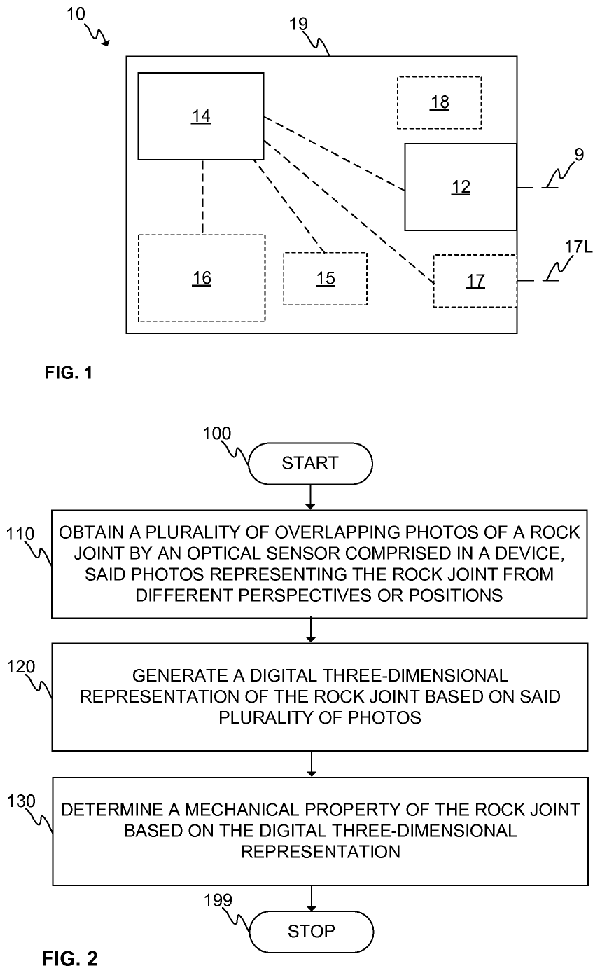

FIG. 1 illustrates schematically a device 10 according to an embodiment of the present invention. The device 10 for estimating a mechanical property of a rock joint may comprise at least one optical sensor 12 for obtaining photos of the rock joint. The optical sensor 12 may be, for example, a digital camera. The device 10 may further comprise control unit 14 in connection with the at least one optical sensor 12. The control unit 14 may preferably comprise a processing unit (not shown in FIG. 1) and, optionally, a memory. The processing unit may advantageously be configured to generate a digital three-dimensional representation of the rock joint based on plurality of at least partly overlapping photos of the rock joint taken with the at least one optical sensor 12. Furthermore, the processing unit may be configured to determine the mechanical property of the rock joint based on the generated digital three-dimensional representation. In FIG. 1, a line of sight related to the optical sensor 12 is marked with reference number 9.

In some embodiments, there may be only one optical sensor 12 or a plurality of optical sensors 12. The plurality of sensors 12 may be arranged with a certain distance, such as 5 or 10 millimetres, from each other.

According to various embodiments, the device 10 may comprise a distance sensor 17, such as an infrared distance sensor. A line of sight related to the distance sensor 17 is marked with reference number 17L.

In embodiments comprising the distance sensor 17, the processing unit may be configured to provide instructions based on measurement data of the distance sensor 17 to an operator via a display 15 of the device 10 for obtaining the plurality of at least partly overlapping photos.

In addition, the device 10 may comprise communication unit 16 for communicating with an external device or system with respect to the device 10. The communication unit 16 may be based on wired or wireless technology. The communication unit 16 may be used to provide a short-range communication connection, such as via BlueTooth™, or an ethernet connection, such as wirelessly or in wired manner.

In addition, the device 10 may comprise electrical power source 18 for providing electrical power to operate the device 10. The electrical power source 18 may be a battery or an electrical connector for connecting an outside power source, such as connection to an electrical grid or a designated electrical power providing device.

Furthermore, the device 10 may comprise a housing 19. In various embodiments, such as described hereinabove with respect to FIG. 1, different elements or features may reside inside the housing 19, thus protected from the environment. However, the housing 19 may advantageously comprise an opening for the optical sensor 12 and an opening for the distance sensor 17, if any.

FIG. 2 illustrates a flow diagram of a method according to an embodiment of the present invention.

Step 100 may refer to a start-up phase of the method. Suitable equipment and components may be obtained, and systems assembled and configured for operation. These may include obtaining or manufacturing a device in accordance with some embodiment of the present invention. Furthermore, necessary communication and/or electrical connections may need to be provided for the systems to operate correctly. Still further, the optical sensor 12, such as the camera, may need to adjusted, for example, with respect to shutter speed and aperture settings.

Step 110 may refer to obtaining a plurality of at least partly overlapping photos of the rock joint by at least one optical sensor 12 comprised in a device 10, wherein said photos represents the rock joint from different perspectives or positions.

The mechanical property may be, for example, roughness, shear strength or friction angle, or any combination thereof.

In some embodiments, a number of said plurality of photos of the rock joint may be at least 20 or at least 40 photos. Alternatively, the number of said plurality of photos may be in the range of 20-40 photos. However, the number of said plurality of photos may be at least 100 which gives even more accurate data for the determination of the mechanical property of the rock joint.

According to an embodiment, the obtaining may comprise arranging the optical sensor 12 such that a first distance between two neighbouring pixels represents a second distance of at most 0.05 centimetres in the rock joint, or in the sample or surface to be studied, in order to achieve submillimetre accuracy.

In some embodiments, the first distance may be estimated based on distance between the optical sensor and the rock joint, focal length, sensor height and/or sensor width, and image height and/or image width, respectively.

Alternatively or in addition, said plurality of photos may represent the rock joint from different angles with respect to the rock joint, such as 10, 20, 45, 60, 75 or 90 degrees.

In various embodiments, the method may comprise moving the optical sensor 12 with respect to the rock joint while the rock joint remains still in its position for obtaining the plurality of photos. In some embodiments, said at least one optical sensor 12 may be comprised in a handheld device, wherein the method comprises moving the handheld device. In another embodiments, said at least one optical sensor 12 may be comprised in an unmanned aerial vehicle, such as a quadcopter, wherein the method comprises moving the unmanned aerial vehicle.

Alternatively, the method may comprise moving the rock joint with respect to said at least one optical sensor 12 while said at least one optical sensor 12 remains still in its position. In some embodiments, the rock joint may be included in a sample arranged on a rotatable platform, wherein the method then comprises moving the rock joint, that is by moving the sample, by the rotatable platform.

In some embodiments, the method may comprise determining overlapping parts of said plurality of photos, and then generating the digital three-dimensional representation of the rock joint based on the overlapping parts.

In various embodiments, the photos may be transmitted to outside the device 10 for processing, such as to a cloud service or an external server. In some embodiments, the results of the processing in the cloud service or the external server may be transmitted back to the device or used as such.

Step 120 may refer to generating a digital three-dimensional representation of the rock joint based on said plurality of photos, optionally, by a control unit 14 of the device 10. Alternatively, step 120 may be performed in the cloud service or in the external server.

In a preferable embodiment, the generation of the digital three-dimensional representation may include determining a point cloud. In various embodiments, the method may comprise removing unnecessary points of the point cloud, such as points outside a portion comprising the rock joint. Still further, with or without said removing of unnecessary parts, the method may comprise performing a triangulation of the point cloud.

According to various embodiments, the obtaining may comprise arranging the optical sensor 12 such that there are 1-120 points, preferably 30-90, most preferably 45-65 points per square millimetre of the rock joint in said plurality of photos in order to achieve submillimetre accuracy. In various embodiments, an accuracy of 0.5 millimetres may be achieved to allow high accuracy joint roughness replication.

Step 130 may refer to determining the mechanical property of the rock joint based on the generated digital three-dimensional representation.

Method execution may be stopped at step 199. The method may be performed once, on demand, continuously, sequentially, or intermittently, for instance. The target rock joint or target surface may be changed between two instances of method execution, for instance.

In an embodiment, the device may comprise a distance sensor 17, such as an infrared distance sensor, and the method may then comprise providing instructions based on measurement data of the distance sensor 17 to an operator via a display 15 of the device 10 for obtaining the plurality of at least partly overlapping photos.

The accuracy of the result may be controlled by adjusting various parameters, such as related to the device 10, the environmental conditions, or to the procedure itself.

Some examples of the device 10 related parameters are as follows: resolution of the optical sensor/camera 12 (such as sensor pixel size), type of the lens, optical sensor 12 related settings (ISO speed or setting, aperture, shutter speed).

Some examples of the environmental conditions related parameters are as follows: the size of the measured object including the rock joint 5, lighting conditions.

Some examples of the procedure related parameters are as follows: distance from camera to the rock joint 5, number of obtained photos, camera intersection angle.

In various embodiments, the control of accuracy may be performed by adjusting at least the optical sensor 12 resolution and the distance between the optical sensor 12 and the rock joint 5.

In various embodiments, a plurality of photos of at least partly overlapping content may be utilized to create or generate 3D point clouds of the rock joint 5, preferably including submillimeter accuracy. In these embodiments, a high-resolution series of photos of convergent and overlapping optical sensor views may be utilized as input.

In various embodiments, a wall sampling distance may, advantageously, be arranged to equal to the inverse of the Nyquist sample frequency for the wall sampling grid. In some embodiments, the wall sample distance of the plurality of photos may be at most 0.05 centimeters per pixel to achieve the submillimeter accuracy.

In some embodiments, the value of the wall sampling distance may be estimated by equation: first distance=MAX(“distance between the optical sensor and the rock joint” *“sensor height”/(“focal length” *“image height”), “distance between the optical sensor and the rock joint” *“sensor width”/(“focal length” *“image width”)).

In various embodiments, a step of pre-processing may be performed to the plurality of obtained at least partly overlapping photos. In the pre-processing step, the photos may be processed, such as cut, so that that all resulting portions of the photos, such as cuts, include substantially only the overlapping portions of the photos, preferably including (the area or portion of the surface comprising) the rock joint 5. Then variance of Laplacians (LAPVs) of the photo binary data may be calculated to estimate the blurriness of the photo cuts. For the calculation, the numerical grey scale values of the photo, i.e., the value component of the photo pixels in the HSV (hue, saturation, value) color model, may be used. Preferably, the processing may be done for cuts of the original set of plurality of photos to select only the parts of the photos content that are in focus.

In some embodiments, The LAPV values may compared with pre-determined values based on long time collection of photo samples. If a photo sample has a LAPV value that is less than the mean value minus two times the standard deviation of the collection, the photo may be removed. The selection criterion may be based on the expectation that the LAPV values follow the Standard Normal Distribution. Therefore, within two standard deviations of the mean accounts for approximately 95% values. However, an additional criteria may be that the photo may be removed only of there is some other photo covering the particular area of the rock joint 5.

The overlap of the photos may be evaluated using the XY-coordinates of the scale-invariant feature transform (SIFT) features found in the photos for determining points that are shared in many photos. This may be estimated in low resolution and with a limited number of calculated features. To obtain better performance, the photos that cover the same area of the rock joint 5 more than a limited time may be removed.

In an embodiment, alternatively or in addition, to obtain even better performance, the photo sets that cover the same area of the rock joint 5 more than m+x times, where m is the minimum number of photos to gain the relative 3D positioning of the photo shot rock surface points, and x is the extra number of photos after the positioning is not essentially improved, can be reduced. The extra photos can be removed randomly or using the LAPV quality estimate of the photos.

In some embodiments, the SIFT detection in the photos may be calculated using the graphics processing unit(s) (GPU(s)) comprised in the control unit of the device 10 or on an external computing system on which the obtained photos may be transmitted. For example, a distributed calculation may be performed by associating photos for each GPU so that the division is in proportion to the computing power of the GPUs.

In various embodiments, full image matching may be utilized in order to find matching features in photos. The full list of combinations of all photos may then be generated. In this stage, pairs of photos which are known to be far of each other can be filtered out. In some embodiments, for example, photos, that have a location information, such as a GPS (Global Positioning System) tag or otherwise provided, that shows distance more than a set value, for example 0.5 metres or 10 degrees or so, between the photo-taking locations or distances in steps in the sequential photo-taking. The resulting list may be divided and sent to the calculating host together with the associated SIFT values and photos.

In some embodiments, a technique for 3D reconstruction using structure from motion may be used to compute the matches. The result may then be written to digital files. When the processing of the matching sets has been performed, the results may be combined, and the estimates of the original optical sensor 12 locations calculated. Thus, a sparse point cloud of the model may be generated. After generating the sparse point cloud, a dense point cloud may be generated, for example, in Polygon File Format.

In some embodiments, noise filtering may be performed on the clouds to reduce the level of floating points. The noise filtering may be performed to remove the points far from the neighbors based on the standard deviation of the distance.

In an embodiment, if the number of the plurality of photos is high, such as over 100 or even over 1000, and there are no cues of their proximity of the photo locations, a copy of the photo set can be scaled to 2-5% of the original size and use the above SIFT and matching for the scaled down data. Then the resulting match data may then be used to create matching par lists for the full-scale photos.

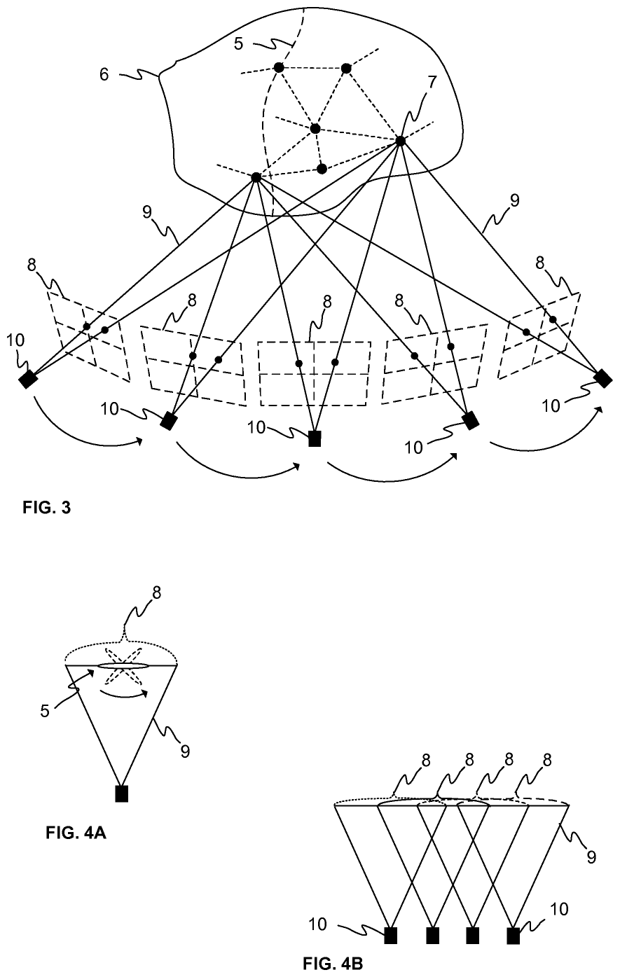

FIG. 3 illustrates schematically a principle for determining the mechanical property of the rock joint 5 in accordance with an embodiment of the present invention. FIG. 3 illustrates a representation of a point cloud 6, which is essentially a set of data points, including various points 7 of the rock joint 5. The points 7 may be determined based on the plurality of photos 8. The photos 8 may be overlapped such that certain relevant points 7 may be included in each or at least part of the photos 8. The plurality of at least overlapping photos 8 may be obtained by moving the optical sensor 12, or the device 10 comprising it, between obtaining of two photos.

In FIG. 3, the optical sensor 12 may be moved between consecutive photos certain number of centimetres or degrees as can be seen in FIG. 3. The movement may be performed, for example, along a semi-circular line. However, in various embodiment, the movement may be performed by a straight line or in some other way to provide different perspectives or positions of the optical sensor 12 with respect to the rock joint 5.

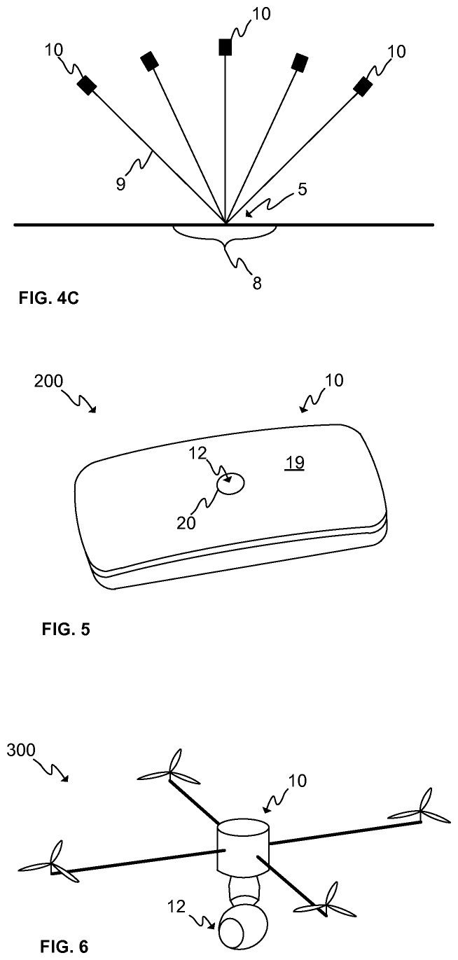

FIGS. 4A-4C illustrate different perspectives or positions of the optical sensor 12 with respect to the rock joint 5 in accordance with some embodiments of the present invention. In FIG. 4A, the optical sensor 12 may be kept in its position and the rock joint 5, or the sample including the rock joint 5, may be rotated to obtain the plurality of at least partly overlapping photos from different perspectives. In FIG. 4B, the optical sensor 12 may be moved along a straight line past the rock joint 5. In FIG. 4C, the movement of the optical sensor 12 may be similar to FIG. 3, however, the optical sensor 12 is moved and arranged such that the rock joint 5 is always directly facing the optical sensor 12.

FIG. 5 illustrates schematically a handheld device 200 according to an embodiment of the present invention. The handheld device 200 may preferably comprise a device 10 in accordance with FIG. 1 and/or an embodiment thereof as described in connection with FIG. 1. Furthermore, in FIG. 5, a housing 19 of the handheld device 200 is illustrated. The housing 19 may define an opening 20 for obtaining photos by the optical sensor 12.

FIG. 6 illustrates schematically an unmanned aerial vehicle 300 according to an embodiment of the present invention. The unmanned aerial vehicle 300 may preferably comprise a device 10 in accordance with FIG. 1 and/or an embodiment thereof as described in connection with FIG. 1, or a handheld device 200 as explained with respect to FIG. 5. Furthermore, in FIG. 6, the unmanned aerial vehicle 300 may comprises means for flight, such as motors for rotating wings, such as known to a skilled person in relation to quadcopters. Furthermore, the unmanned aerial vehicle 300 preferably comprises means for controlling the movement of the vehicle 300, such as by predetermining the flight route, or by a remote control (not shown) as is known to a skilled person.

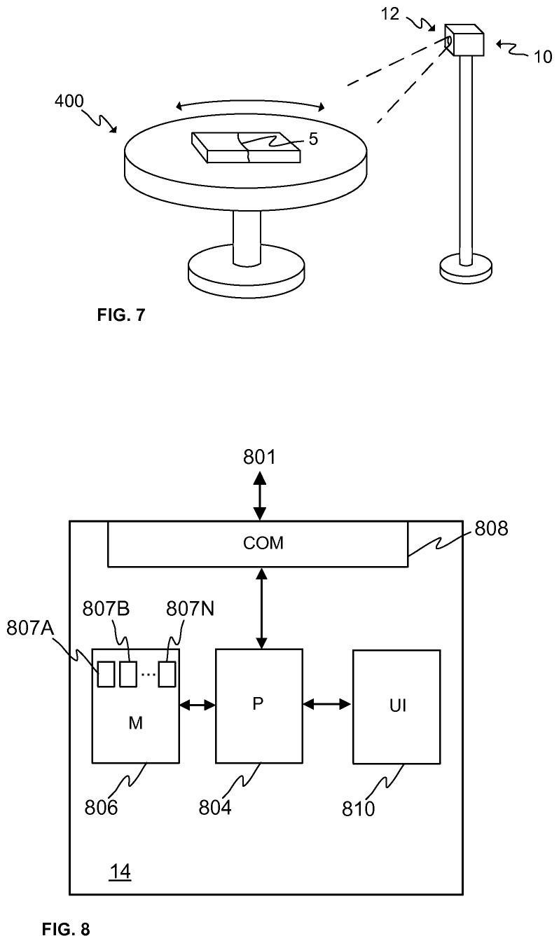

FIG. 7 illustrates schematically a combination of a device 10 and a rotatable platform 400 according to an embodiment of the present invention. The rotatable platform 400 may comprise means for rotating the platform, such as a motor, e.g. a servomotor, and means for providing electrical power to the motor. The platform may be arranged to rotated in controlled manner, such as certain number of degrees at a time, that is between two photos. Thus, the rotatable platform 400, or at least the controlling unit thereof, and the control unit 14 of the device 10 may be arranged to be in communication with each other.

FIG. 8 illustrates schematically a control unit 14 according to an embodiment of the present invention. External units 801 may be connected to a communication interface 808 of the control unit 14. External unit 801 may comprise wireless connection or a connection by a wired manner. The communication interface 808 may provide interface for communication with external units 801, such as the controlling means for adjusting or operating the optical sensor 12, or the related elements, for example, the shutter thereof. There may also be connecting to the external system, such as a laptop or a handheld device 200 or an unmanned aerial device 300 or a rotatable platform 400, respectively. There may also be a connection to a database of the device 10 or an external database including information used in controlling the operation of the device 10.

The control unit 14 may comprise one or more processors 804, one or more memories 806 being volatile or non-volatile for storing portions of computer program code 807A-807N and any data values and possibly one or more user interface units 810. The mentioned elements may be communicatively coupled to each other with e.g. an internal bus.

The processor 804 of the control unit 14 may be at least configured to implement at least some method steps as described hereinbefore, such as in connection with FIG. 2. The implementation of the method may be achieved by arranging the processor 804, that is the processing unit, to execute at least some portion of computer program code 807A-807N stored in the memory 806 causing the processor 804, and thus the control unit 14, to implement one or more method steps as described hereinbefore. The processor 804 may thus be arranged to access the memory 806 and retrieve and store any information therefrom and thereto. For sake of clarity, the processor 804 herein refers to any unit suitable for processing information and control the operation of the device 10, among other tasks, such as also the movement of the unmanned aerial vehicle 300 or the rotatable platform 400. The operations may also be implemented with a microcontroller solution with embedded software. Similarly, the memory 806 is not limited to a certain type of memory only, but any memory type suitable for storing the described pieces of information may be applied in the context of the present invention.

The specific examples provided in the description given above should not be construed as limiting the applicability and/or the interpretation of the appended claims. Lists and groups of examples provided in the description given above are not exhaustive unless otherwise explicitly stated.

1. A method for estimating a mechanical property of a rock joint, the method comprising:
obtaining a plurality of at least partly overlapping photos of the rock joint by at least one optical sensor comprised in a device, said photos representing the rock joint from different perspectives or positions,
generating a digital three-dimensional representation of the rock joint based on said plurality of photos, and
determining the mechanical property of the rock joint based on the generated digital three-dimensional representation.

2. The method according to claim 1, wherein the mechanical property is roughness, shear strength or friction angle, or any combination thereof.

3. The method according to claim 1, wherein a number of said plurality of photos of the rock joint is at least 20 or at least 40 photos, or in the range of 20-40 photos.

4. The method according to claim 1, wherein the generation of the digital three-dimensional representation includes determining a point cloud.

5. The method according to claim 1, wherein said plurality of photos represent the rock joint from different angles with respect to the rock joint.

6. The method according to claim 1, wherein the device comprises a distance sensor, such as an infrared distance sensor, and the method comprises providing instructions based on measurement data of the distance sensor to an operator via a display of the device for obtaining the plurality of at least partly overlapping photos.

7. The method according to claim 1, comprising moving the at least one optical sensor with respect to the rock joint while the rock joint remains still in its position.

8. The method according to claim 7, wherein said at least one optical sensor is comprised in a handheld device, and the method comprises moving the handheld device.

9. The method according to claim 7, wherein said at least one optical sensor is comprised in an unmanned aerial vehicle, such as a quadcopter, and the method comprises moving the unmanned aerial vehicle.

10. The method according to claim 1, comprising moving the rock joint with respect to said at least one optical sensor while said at least one optical sensor remains still in its position.

11. The method according to claim 10, wherein the rock joint is included in a sample arranged on a rotatable platform, and the method comprises moving the rock joint by the rotatable platform.

12. The method according to claim 1, comprising determining overlapping parts of said plurality of photos, and generating the digital three-dimensional representation of the rock joint based on the overlapping parts.

13. The method according to claim 4, comprising removing unnecessary points of the point cloud, such as points outside a portion comprising the rock joint.

14. The method according to claim 13, wherein the obtaining a plurality of at least partially overlapping photos comprises arranging the optical sensor such that there are 1-120 points, preferably 30-90, most preferably 45-65 points per square millimetre of the rock joint, such as with respect to a sample including the rock joint, in said plurality of photos.

15. The method according to claim 4, comprising performing a triangulation of the point cloud.

16. A device for estimating a mechanical property of a rock joint, wherein the device comprises at least one optical sensor for obtaining photos of the rock joint, and a control unit in connection with the at least one optical sensor, wherein the control unit comprises a processing unit configured to
generate a digital three-dimensional representation of the rock joint based on plurality of at least partly overlapping photos of the rock joint, and
determine the mechanical property of the rock joint based on the generated digital three-dimensional representation.

17. The device according to claim 16, comprising a distance sensor, such as an infrared distance sensor, wherein the processing unit is configured to provide instructions based on measurement data of the distance sensor to an operator via a display of the device for obtaining the plurality of at least partly overlapping photos.

18. A handheld device comprising the device according to claim 16.

19. An unmanned aerial vehicle, such as a quadcopter, comprising the device according to claim 16.

20. A combination of a rotatable platform and the device according to claim 16.
